(19) United States
(12) Patent Application Publication
(10) Pub. No.: US 2025/0161710 A1
(43) Pub. Date: May 22, 2025

(54) HEAD ACUPOINT LASER STIMULATOR

(71) Applicant: UNIVERSITY-INDUSTRY COOPERATION GROUP OF KYUNG HEE UNIVERSITY, Yongin-si, Gyeonggi-do (KR)

(72) Inventors: Seung Hun CHO, Seoul (KR); Yunna KIM, Seoul (KR)

(73) Assignee: UNIVERSITY-INDUSTRY COOPERATION GROUP OF KYUNG HEE UNIVERSITY, Yongin-si, Gyeonggi-do (KR)

(21) Appl. No.: 18/840,475

(22) PCT Filed: Jan. 19, 2023
(86) PCT No.: PCT/KR2023/000903

§ 371 Date: Aug. 21, 2024

(30) Foreign Application Priority Data
Feb. 21, 2022 (KR) 10-2022-0022308

(51) Int. Cl. A61N 5/067; A61N 5/06

(52) U.S. Cl. CPC A61N 5/067; A61N 5/0619; A61N 2005/0626; A61N 2005/0632; A61N 2005/0647; A61N 2005/0659; A61N 2005/0662

(57) ABSTRACT

The present invention provides a head acupoint laser stimulator including a case portion that is formed in a dome shape; a plurality of laser generation units which are disposed inside the case portion, and emit laser needles to stimulate acupoints distributed on the head, and in which a plurality of lasers are disposed to be separated from each other; and a laser control unit that controls the plurality of laser generation units. According to the head acupoint laser stimulator of the present invention, the laser needles generated from the plurality of laser generation units are emitted to stimulate the acupoints distributed on the head, thereby affecting the meridian system of the body to show a therapeutic effect similar to needling.

### TECHNICAL FIELD

The present invention relates to a head acupoint laser stimulator, and more specifically, to a head acupoint laser stimulator that stimulates the scalp by irradiating the scalp with laser light.

### BACKGROUND

Acupuncture treatment using needles to stimulate acupoints distributed on the head is known to be effective for nervous system diseases and mental disorders, and can also be applied to otolaryngology diseases, internal medicine diseases, surgical diseases, dermatology diseases, and musculoskeletal diseases. The effects follow classical treatment principles and are also explained as treating the disease by needling a portion corresponding to the head based on the cerebral cortex theory of western medicine.

In acupuncture treatment, the acupoints on the meridians that pass through the head (Leg Greater Yang Bladder Meridian, Leg Lesser Yang Gallbladder Meridian, Leg Yangmyeong Stomach Meridian, Governor Vessel Meridian, and Arm Lesser Yang Triple Warmer Meridian), extra acupoints distributed on the head, and the like are used.

However, since the scalp layer on the head is thin and the skull is located on the head, a general fine needle falls off quickly, making it difficult to needle. Therefore, in clinical practice, the needling is done at an angle to solve these shortcomings, but it is not easy to stimulate the correct acupoint, and it increases the possibility of bleeding, bruising, etc., which can increase the patient's discomfort.

In the 1990s, it was reported that irradiating blood vessels with low-power laser has beneficial effects on ischemic cerebrovascular diseases, neurological diseases, cardiovascular diseases, urinary system diseases, ophthalmic diseases, diabetes, skin diseases, and arthritis. From then on, the treatment method of irradiating the blood vessels with low-power laser has been called LLLT (Low Level Laser Therapy).

Through non-invasive treatment, there is no invasive infection compared to regular acupuncture and the process of disinfection can be reduced, so it can be performed stably. In addition, it has the advantage of being able to accurately and quantitatively apply the amount of light irradiated to acupoints, enabling uniform treatment, and increasing the effect when applied together with treatment of a different mechanism from general acupuncture treatment.

In order to achieve the same effect as acupuncture without causing damage to the human body by varying the wavelength and output of the laser, research is being conducted on different wavelengths and outputs, among which is Low Level Laser Therapy ((LLLT), hereinafter referred to as LLLT) is being studied most actively.

However, conventional laser needle has only been developed in a form that can stimulate only one area. Considering the general form of acupuncture treatment, which stimulates multiple acupoints simultaneously for 10 to 20 minutes during acupuncture treatment, there is a problem that it is difficult to apply in clinical practice.

Prior art for the present invention may include Korea Registered Patent No. 10-1009462 (title of the invention: phototherapeutic apparatus).

### SUMMARY OF INVENTION

### Technical Problem

The present invention is created to solve the above problems, and an object of the present invention is to provide a head acupoint laser stimulator that operates and controls all or part of a plurality of lasers according to the user's symptoms by arranging the plurality of lasers along the acupoints distributed on the head.

### Technical Solution

The present invention provides a head acupoint laser stimulator including a case portion that is formed in a dome shape; a plurality of laser generation units which are disposed inside the case portion, and emit laser needles to stimulate acupoints distributed on the head, and in which a plurality of lasers are disposed to be separated from each other; and a laser control unit that controls the plurality of laser generation units.

The plurality of laser generation units may include first to sixth lasers each stimulates a first stimulation portion that stimulates Leg Greater Yang Bladder Meridian including Acupoints including Tongcheon, Nakgak, Okchim, Cheonchu, Ocheo, Gokcha, Michung, Chanjuk, Jeongmyeong, and Seunggwang, a second stimulation portion that stimulates Governor Vessel Meridian including Shinhoe, Sangseong, Sinjeong, Jeonjeong, Soyo, Sugu, Taedan, Baekhoe, Hujeong, Gangae, Noeho, Pungbu, and Amun, a third stimulation portion that stimulates Leg Lesser Yang Gallbladder Meridian including Pungji, Wangol, Noegong, Bubaek, Gyuum, Cheonchung, Solgok, Seungryeong, Jeongyeong, Hyeonro, Mokchang, Bonsin, Duimup, Hamyeom, Yangbaek, Hyeonri, Gokbin, Sangkan, Tongjarou, and Cheonghoe, a fourth stimulation portion that stimulates Leg Yangmyeong Stomach Meridian including Duyu, Hagwan, Hyeopcha, Daeyeong, Seungeup, Sabaek, Jichang, and Georyo, a fifth stimulation portion that stimulates Arm Lesser Yang Triple Warmer Meridian including Cheonyu, Yepung, Gyemaek, Nosik, Gakson, Sajukgong, Hwaryo, and Imun, and a sixth stimulation portion that stimulates extra acupoints of acupoints including Dangyang, Sasinchong, Indang, Sangyeonghyang, Eo, Taeyang, Guhu, Yicheom, and Yemyeong.

The first stimulation portion may be a plurality of laser stimulators that are formed at regular intervals from a front side to a back side of the case portion along an imaginary line formed by being separated to one side of a center line of the case portion.

The second stimulation portion may be a plurality of laser stimulators that are formed at regular intervals from a front side to a back side of the case portion along a center line of the case portion.

The third stimulation portion may be a plurality of laser stimulators that are formed in a right hemisphere of the case portion, and disposed from a back bottom of the case portion to a front top of the case portion separated from each other along an inner surface of an outer circumference of the case portion.

According to the head acupoint laser stimulator according to the present invention, it may further include an auxiliary case portion which is installed on a right side of the case portion, and in which a plurality of lasers emitting laser needles to stimulate acupoints distributed on the face are disposed with being separated from each other, in which the fourth stimulation portion may be a plurality of laser stimulators that are formed at regular intervals from a front side to a back side of the auxiliary case portion.

The fifth stimulation portion may be a plurality of laser stimulators that are disposed from a center of a side of the case portion to a front top of the case portion separated from each other along an inner surface of an outer circumference of the case portion.

The sixth stimulation portion may be a plurality of laser stimulators that are disposed from an upper center of the case portion to the front of the case portion separated from each other along an inner surface of an outer circumference of the case portion.

The laser control unit may control the first to fourth lasers to stimulate the entire first to fourth stimulation portions.

The laser control unit may control the first to fourth lasers to stimulate one of the first to fourth stimulation portions.

According to the head acupoint laser stimulator according to the present invention, it may further include a connection module that is connected wired or wirelessly to the laser control unit and a smartphone of a user, in which the plurality of laser generation units may be connected to the smartphone of the user by the connection module, and the intensity, stimulation position, and time of the laser needle may be adjusted using the smartphone.

The connection module may be connected to a smartphone of a medical staff member at a remote distance, and the medical staff may control the intensity, stimulation position, and time adjustment of the laser needle using the smartphone of the medical staff member.

The plurality of lasers may emit laser needles of visible light and infrared light. The plurality of lasers may emit laser needles of an intensity of 1 mv to 50 mv.

### Advantageous Effects

According to the head acupoint laser stimulator of the present invention, the laser needles generated from the plurality of laser generation units are emitted to stimulate the acupoints distributed on the head, thereby affecting the meridian system of the body to show a therapeutic effect similar to needling.

In addition, the laser needle not only ensures safety as a non-pharmacological treatment, but also is a non-contact procedure, so there is no pain or bleeding during the treatment for the user. Since the laser needle is a device that excludes a physical metal needle, it can solve the risk of infection of other diseases, and thereby there is an advantage of providing luxury and comfort to the user at the same time.

### BRIEF DESCRIPTION OF THE DRAWINGS

FIG. 1 is a view illustrating a first stimulation portion on a head acupoint.

FIG. 2 is a view illustrating a second stimulation portion on the head acupoint.

FIG. 3 is a view illustrating a third stimulation portion on the head acupoint.

FIG. 4 is a view illustrating a fourth stimulation portion on the head acupoint.

FIG. 5 is a view illustrating a fifth stimulation portion on the head acupoint.

FIG. 6 is a view illustrating a sixth stimulation portion on the head acupoint.

FIG. 7 is a bottom perspective view of a head acupoint laser stimulator of the present invention.

FIG. 8 is a plan view of FIG. 7.

FIG. 9 is a view illustrating a usage status of the head acupoint laser stimulator according to a first embodiment of the present invention.

FIG. 10 is a view illustrating a usage status of the head acupoint laser stimulator according to a second embodiment of the present invention.

FIG. 11 is a view illustrating a user controlling the head acupoint laser stimulator using a smartphone.

### BEST MODE FOR INVENTION

The present invention is described with reference to the embodiments illustrated in the drawings, but these are merely exemplary, and those skilled in the art will understand that various modifications and equivalent other embodiments are possible therefrom. Therefore, the true scope of technical protection of the present invention should be determined by the technical spirit of the attached claims.

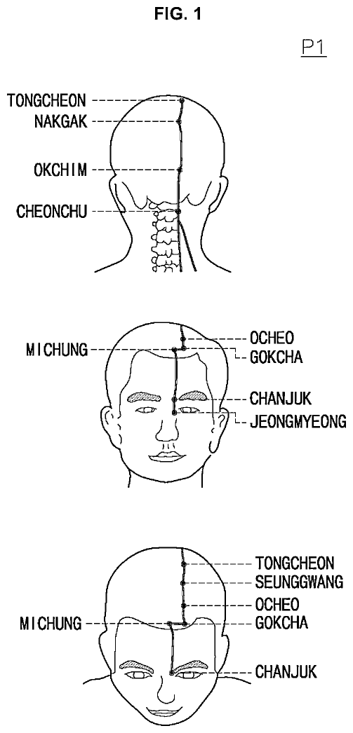

Referring to FIGS. 1 to 4, first to fourth stimulation portions P1 to P4 that stimulate Leg Greater Yang Bladder Meridian, Governor Vessel Meridian, Leg Lesser Yang Gallbladder Meridian, and Leg Yangmyeong Stomach Meridian among the acupoints distributed in the head, respectively, are illustrated.

FIG. 1 illustrates the first stimulation portion P1 that stimulates Leg Greater Yang Bladder Meridian among the acupoints distributed in the head, and includes Tongcheon, Nakgak, Okchim, Cheonchu, Ocheo, Gokcha, Michung, Chanjuk, Jeongmyeong, and Seunggwang.

The Leg Greater Yang Bladder Meridian is connected to the kidney, bladder, brain, eyes, ears, nape, spine, buttocks, back of the leg, little toe, etc., and when the Leg Greater Yang Bladder Meridian is stimulated, it can alleviate symptoms such as eye and head pain, back pain, hip joint, knee joint and calf pain, hemorrhoids, hemorrhoids, tremor, runny nose, nosebleeds, and inability to use the little toe.

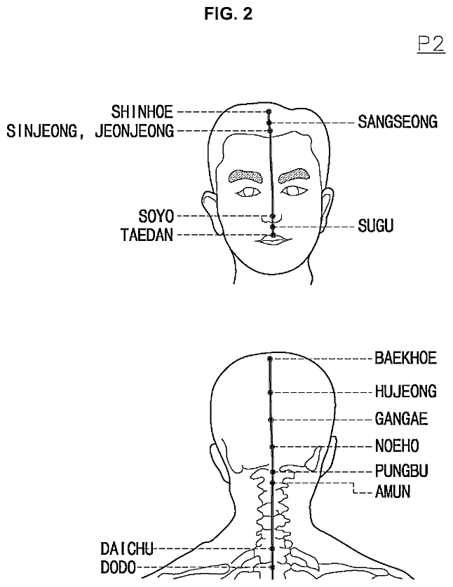

FIG. 2 illustrates the second stimulation portion P2 that stimulates Governor Vessel Meridian among the acupoints distributed in the head, and includes Shinhoe, Sangseong, Sinjeong, Jeonjeong, Soyo, Sugu, Taedan, Baekhoe, Hujeong, Gangae, Noeho, Pungbu, and Amun. Includes.

The Governor Vessel Meridian is connected to the brain, spine, and perineum, and when the Governor Vessel Meridian is stimulated, it can relieve symptoms such as stiff and painful spine and heavy head symptoms.

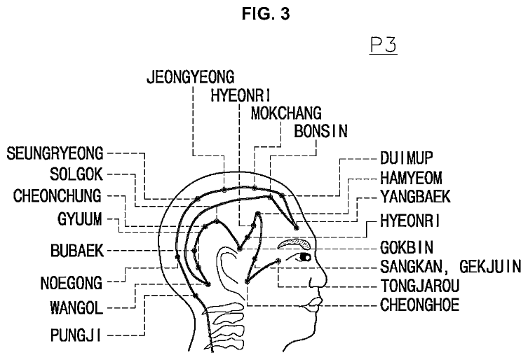

FIG. 3 illustrates the third stimulation portion P3 that stimulates Leg Lesser Yang Gallbladder Meridian among the acupoints distributed in the head, and includes Pungji, Wangol, Noegong, Bubaek, Gyuum, Cheonchung, Solgok, Seungryeong, Jeongyeong, Hyeonro, Mokchang, Bonsin, Duimup, Hamyeom, Yangbaek, Hyeonri, Gokbin, Sangkan, Tongjarou, and Cheonghoe.

The Leg Lesser Yang Gallbladder Meridian is mainly distributed on the sides of the body and is connected to the gallbladder, liver, eyes, ears, side of the head, sacrum, hip joint, outer leg, fourth toe, and big toe. When the Leg Lesser Yang Gallbladder Meridian is stimulated, it can relieve symptoms such as bitterness in mouth, frequent sighing, chills, fever, vomiting, sweating, migraines, and pain on eye strain, supraclavicular fossa, armpits, flanks, hip joints, ant area, steel area, and the outside of the legs.

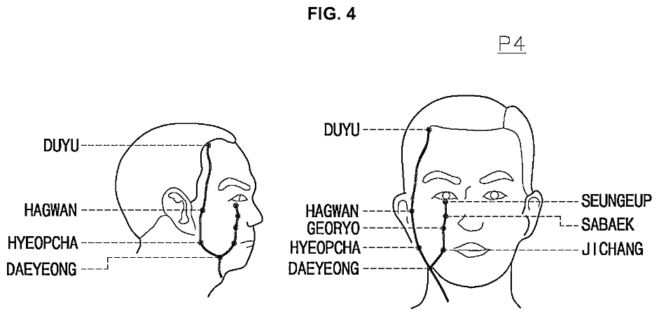

FIG. 4 illustrates the fourth stimulation portion P4 that stimulates Leg Yangmyeong Stomach Meridian among the acupoints distributed in the head, and includes Duyu, Hagwan, Hyeopcha, Daeyeong, Seungeup, Sabaek, Jichang, and Georyo.

The Leg Yangmyeong Stomach Meridian is connected to the stomach, rain, eyes, nose, mouth, upper two teeth, mandibular joint, forehead, throat, breast, front foot of the leg, and the first, second, and third toes. When the Leg Yangmyeong Stomach Meridian is stimulated, it can relieve symptoms such as high fever, irritability, sweating, impaired consciousness, delirium tremens, mania, sore throat, epistaxis, lip march, occipital pain, breast pain, knee pain, and digestive system disorders.

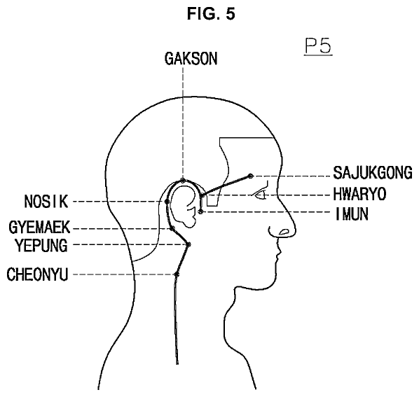

FIG. 5 illustrates the fifth stimulation portion P5 that stimulates Arm Lesser Yang Triple Warmer Meridian among the acupoints distributed in the head, and includes Cheonyu, Yepung, Gyemaek, Nosik, Gakson, Sajukgong, Hwaryo, and Imun.

The Arm Lesser Yang Triple Warmer Meridian starts from the tip of the fourth finger, passes through the fourth finger bone, passes between the fourth and fifth iliac bones, passes through Yangji acupuncture point of the wrist, the middle of the outer arm, the elbow, the middle of the outer arm, and connects to the shoulder. When the Arm Lesser Yang Triple Warmer Meridian is stimulated, it can relieve symptoms such as difficulty hearing, swollen tonsils, sweating, pain in the corners of the eyes, cheeks, behind the ears, shoulders, and the outside of the arms.

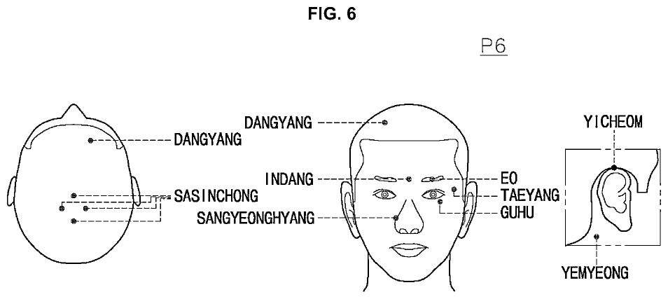

FIG. 6 illustrates the sixth stimulation portion P6 that stimulates extra acupoints among the acupoints distributed in the head, and includes Dangyang, Sasinchong, Indang, Sangyeonghyang, Eo, Taeyang, Guhu, Yicheom, and Yemyeong.

The extra acupoints are also called Gihyeol, Gyeongoehyeol. They are acupoints that do not belong to the fourteen meridians. The extra acupoints are constantly being replenished based on experience gained during clinical treatment, and their number now exceeds 1,500. In a broad sense, new acupoints that have been newly replenished in recent years are included in the extra acupoints.

Here, the action and indications of the extra acupoints cannot be interpreted based on the meridian theory, and the method of determining the location is unclear and is held in different ways depending on the people who find the acupoint. Although the indications for the above-mentioned extra acupoints are simple, there are many acupoints with high therapeutic efficacy, and when the extra acupoints are stimulated, it can relieve symptoms such as headache, anxiety, myopia, hyperopia, rhinitis, laryngitis, asthma, and insomnia.

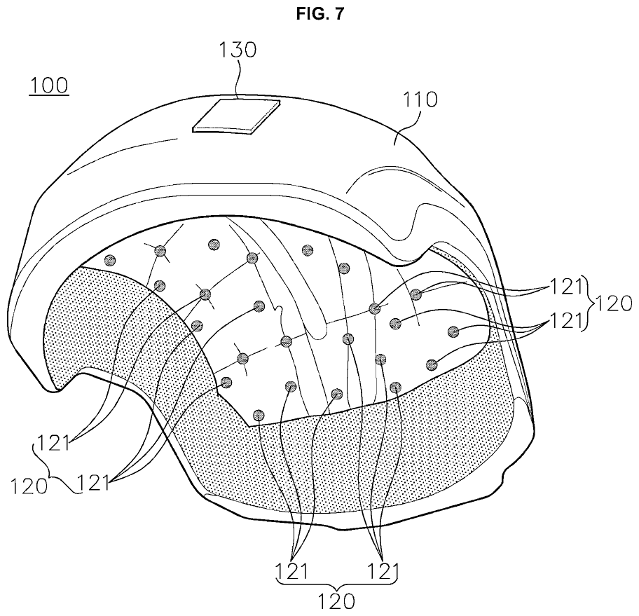

Referring to FIG. 7, a head acupoint laser stimulator 100 according to an embodiment of the present invention will be described.

The head acupoint laser stimulator 100 includes a case portion 110, a plurality of laser generation units 120, and a laser control unit 130.

The case portion 110 is formed in a dome shape and is disposed on the head of a user H in order to stimulate the acupoints distributed on the head.

The plurality of laser generation units 120 are disposed inside the case portion 110, and emit laser needles to stimulate the acupoints distributed on the head, and a plurality of lasers 121 are disposed therein to be separated from each other. In addition, in the plurality of laser generation units 120, first to fourth lasers (not illustrated) are disposed to stimulate the first to fourth stimulation portions P1 to P4, respectively.

Here, the plurality of lasers 121 emit laser needles of visible light and infrared light, and at this time, the emitted laser needles are emitted at an intensity of 1 mv to 50 mv.

The laser control unit 130 controls the intensity, stimulation position, time, etc. of the laser needles emitted from the plurality of laser generation units 121. In addition, the laser control unit 130 controls the first to fourth lasers to stimulate the entire first to fourth stimulation portions P1 to P4 or stimulate one of the stimulation portions P1 to P4 which is selected by the user H.

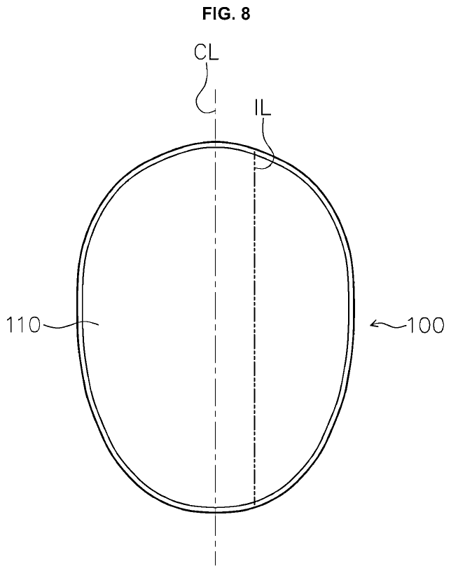

Referring to FIGS. 8 to 10, the head acupoint laser stimulator 100 of the first and second embodiments of the present invention is illustrated, and a method for stimulating the first to fourth stimulation portions P1 to P4 will be described.

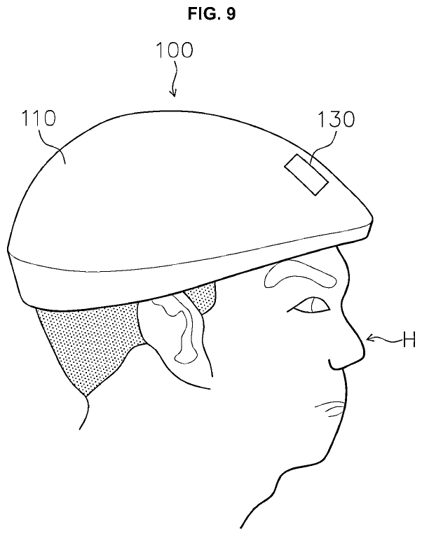

Referring to FIGS. 8 and 9, using the first embodiment of the present invention, the first to third stimulation portions P1 to P3, the fifth stimulation portion P5, and the sixth stimulation portion P6 can be stimulated.

In the first stimulation portion P1, the plurality of lasers 121 are formed at regular intervals from the front side to the back side of the case portion 110 along an imaginary line IL formed by being separated to one side of a center line CL of the case portion 110 to stimulate the first stimulation portion P1.

Here, the first stimulation portion P1 stimulates Michung, Gokcha, Ocheo, Seunggwang, Tongcheon, Nakgak, and Okchim that are spaced to the left side about the head of the user H and connected to the back side of the head, thereby relieving symptoms and pain corresponding to each acupoint.

In the second stimulation portion P2, the plurality of lasers 121 are formed at regular intervals from the front side to the back side of the case portion 110 along a center line CL of the case portion 110 to stimulate the second stimulation portion P2.

Here, the second stimulation portion P2 is located at the front of the head of the user H. By stimulates Shinhoe, Sangseong, Sinjeong, Baekhoe, Hujeong, Gangae, and Noeho connected from the front side to the back side of the user H to relieve symptoms and pain corresponding to each acupoint.

The third stimulation portion P3 is formed in the right hemisphere of the case portion 110, and the plurality of lasers 121, which are disposed from the back bottom of the case portion 110 to the front top of the case portion 110 separated from each other along an inner surface of an outer circumference of the case portion 110, are formed to stimulate the third stimulation portion P3.

Here, the third stimulation portion P3 stimulates Pungji, Wangol, Noegong, Bubaek, Gyuum, Cheonchung, Solgok, Seungryeong, Jeongyeong, Hyeonro, Mokchang, Bonsin, Duimup, Hamyeom, Yangbaek, Hyeonri, Gokbin, Sangkan, Tongjarou, and Cheonghoe which are formed in the lower back, side, and upper eyebrow, the front, back, and upper side of the right ear, and right of middle of the forehead based on the right side of the head of the user H to relieve symptoms and pain corresponding to each acupoint.

In the fifth stimulation portion P5, the plurality of lasers 121, which are disposed from the center of the side of the case portion 110 to the front top of the case portion 110 separated from each other along the inner surface of the outer circumference of the case portion 110, are formed to stimulate the fifth stimulation portion P5.

Here, the fifth stimulation portion P5 stimulates Yepung, Gyemaek, Nosik, Gakson, Hwaryo, Imun, and Sajukgong which are formed in on the upper, lower, front, and back sides of the right ear, and the upper right eyebrow, based on the right side of the head of the user H to relieve symptoms and pain corresponding to each acupoint.

In the sixth stimulation portion P6, the plurality of lasers 121, which are disposed from the upper center of the case portion 110 to the front of the case portion 110 separated from each other along the inner surface of the outer circumference of the case portion 110, are formed to stimulate the sixth stimulation portion P6.

Here, the sixth stimulation portion P6 stimulates Sasinchong and Dangyang disposed at the center of the head of the user H and the front left side of the head of the user H to relieve the symptoms and pain corresponding to each acupoint.

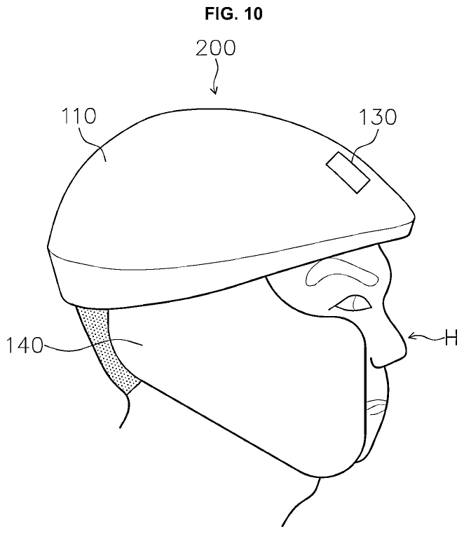

Referring to FIG. 10, using the second embodiment of the present invention, the first stimulation portion P1 to the sixth stimulation portion P6 can be selectively stimulated, and a method for stimulating the fourth stimulation portion P4 excluding the first stimulation portion to the third stimulation portion P3, the fifth stimulation portion P5, and the sixth stimulation portion P6 will be described.

A head acupoint laser stimulator 200 according to the second embodiment of the present invention includes the case portion 110, a plurality of laser generation units 120, the laser control unit 130, and an auxiliary case portion 140.

The case portion 110, the plurality of laser generation units 120, and the laser control unit 130 are the same as the configurations of the head acupoint laser stimulator 100 according to the first embodiment of the present invention mentioned above, and hereinafter, the contents of the case portion 110, the plurality of laser generation units 120, and the laser control unit 130 will be omitted.

The auxiliary case portion 140 is installed on the right side of the case portion 110, and the plurality of lasers 121 that emit laser needles to stimulate the acupoints distributed on the face are disposed inside separated from each other. Additionally, by installing the auxiliary case portion 140, the fourth stimulation portion P4 is stimulated.

In the fourth stimulation portion P4, the plurality of lasers 121 are formed at regular intervals from the front side to the back side of the auxiliary case portion 140 to stimulate the fourth stimulation portion P4.

Here, the fourth stimulation portion P4 is connected to the chin from the bottom of the right eye, is connected to the bottom of the right ear and forward, and then connected to the top of the head based on the right side of the head of the user H. Duyu, Hagwan, Hyeopcha, Daeyeong, Seungeup, Sabaek, Jichang, and Georyo corresponding thereto are stimulated to relieve the symptoms and pain corresponding to each acupoint.

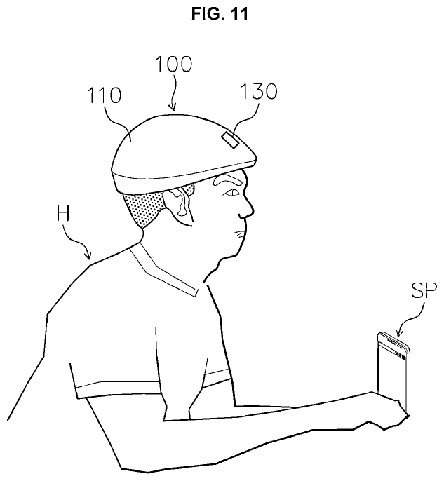

Referring to FIG. 11, the head acupoint laser stimulator 100 is disposed with a connection module (not illustrated) to be connected wired or wirelessly for connecting the laser control unit 130 and the smartphone SP of the user H to control the smartphone SP of the user H and the head acupoint laser stimulator 100 by wired or wireless connection and remotely.

The plurality of laser generation units 120 are connected to the smartphone SP of the user H by the connection module, and the intensity, stimulation position, and time of the laser needle are adjusted using the smartphone SP.

In addition, the connection module is connected to a smartphone of a medical staff member at a remote distance, and the medical staff can perform remote medical treatment by controlling the intensity, stimulation position, and time adjustment of the laser needle using the smartphone of the medical staff member.

The laser needle emitted from the head acupoint laser stimulator 100 according to an embodiment of the present invention is a type of meridian stimulation therapy that irradiates laser light to the acupoints and affects the meridian system of the body through energy stimulation, thereby illustrating a therapeutic effect similar to needling using existing needles.

The laser needle not only ensures safety as a non-pharmacological treatment, but also is a non-contact procedure, so there is no pain or bleeding during the treatment for the user. Since the laser needle is a device that excludes a physical metal needle, it can solve the risk of infection of other diseases, and thereby there is an advantage of providing luxury and comfort to the user at the same time.

While laser treatment using a conventional laser directly irradiates the skin or deep lesion tissue with the laser to induce a direct effect in the lesion tissue, the laser needle stimulates meridians such as acupoints, extra acupoints, and Ashi points of the human body with the laser light source to induce a therapeutic effect through the function of the meridians.

1. A head acupoint laser stimulator comprising:
a case portion that is formed in a dome shape;
a plurality of laser generation units which are disposed inside the case portion, and emit laser needles to stimulate acupoints distributed on the head, and in which a plurality of lasers are disposed to be separated from each other; and
a laser control unit that controls the plurality of laser generation units.

2. The head acupoint laser stimulator of claim 1, wherein the plurality of laser generation units includes first to sixth lasers each stimulates
a first stimulation portion that stimulates Leg Greater Yang Bladder Meridian including Acupoints including Tongcheon, Nakgak, Okchim, Cheonchu, Ocheo, Gokcha, Michung, Chanjuk, Jeongmyeong, and Seunggwang,
a second stimulation portion that stimulates Governor Vessel Meridian including Shinhoe, Sangseong, Sinjeong, Jeonjeong, Soyo, Sugu, Taedan, Baekhoe, Hujeong, Gangae, Noeho, Pungbu, and Amun,
a third stimulation portion that stimulates Leg Lesser Yang Gallbladder Meridian including Pungji, Wangol, Noegong, Bubaek, Gyuum, Cheonchung, Solgok, Seungryeong, Jeongyeong, Hyeonro, Mokchang, Bonsin, Duimup, Hamyeom, Yangbaek, Hyeonri, Gokbin, Sangkan, Tongjarou, and Cheonghoe,
a fourth stimulation portion that stimulates Leg Yangmyeong Stomach Meridian including Duyu, Hagwan, Hyeopcha, Daeyeong, Seungeup, Sabaek, Jichang, and Georyo,
a fifth stimulation portion that stimulates Arm Lesser Yang Triple Warmer Meridian including Cheonyu, Yepung, Gyemaek, Nosik, Gakson, Sajukgong, Hwaryo, and Imun, and
a sixth stimulation portion that stimulates extra acupoints of acupoints including Dangyang, Sasinchong, Indang, Sangyeonghyang, Eo, Taeyang, Guhu, Yicheom, and Yemyeong.

3. The head acupoint laser stimulator of claim 2, wherein the first stimulation portion is a plurality of laser stimulators that are formed at regular intervals from a front side to a back side of the case portion along an imaginary line formed by being separated to one side of a center line of the case portion.

4. The head acupoint laser stimulator of claim 2, wherein the second stimulation portion is a plurality of laser stimulators that are formed at regular intervals from a front side to a back side of the case portion along a center line of the case portion.

5. The head acupoint laser stimulator of claim 2, wherein the third stimulation portion is a plurality of laser stimulators that are formed in a right hemisphere of the case portion, and disposed from a back bottom of the case portion to a front top of the case portion separated from each other along an inner surface of an outer circumference of the case portion.

6. The head acupoint laser stimulator of claim 2, further comprising:
an auxiliary case portion which is installed on a right side of the case portion, and in which a plurality of lasers emitting laser needles to stimulate acupoints distributed on the face are disposed with being separated from each other,
wherein the fourth stimulation portion is a plurality of laser stimulators that are formed at regular intervals from a front side to a back side of the auxiliary case portion.

7. The head acupoint laser stimulator of claim 2, wherein the fifth stimulation portion is a plurality of laser stimulators that are disposed from a center of a side of the case portion to a front top of the case portion separated from each other along an inner surface of an outer circumference of the case portion.

8. The head acupoint laser stimulator of claim 2, wherein the sixth stimulation portion is a plurality of laser stimulators that are disposed from an upper center of the case portion to the front of the case portion separated from each other along an inner surface of an outer circumference of the case portion.

9. The head acupoint laser stimulator of claim 2, wherein the laser control unit controls the first to fourth lasers to stimulate the entire first to fourth stimulation portions.

10. The head acupoint laser stimulator of claim 2, wherein the laser control unit controls the first to fourth lasers to stimulate one of the first to fourth stimulation portions.

11. The head acupoint laser stimulator of claim 1, further comprising:
a connection module that is connected wired or wirelessly to the laser control unit and a smartphone of a user,
wherein the plurality of laser generation units are connected to the smartphone of the user by the connection module, and the intensity, stimulation position, and time of the laser needle are adjusted using the smartphone.

12. The head acupoint laser stimulator of claim 11, wherein the connection module is connected to a smartphone of a medical staff member at a remote distance, and the medical staff controls the intensity, stimulation position, and time adjustment of the laser needle using the smartphone of the medical staff member.

13. The head acupoint laser stimulator of claim 1, wherein the plurality of lasers emit laser needles of visible light and infrared light.

14. The head acupoint laser stimulator of claim 1, wherein the plurality of lasers emit laser needles of an intensity of 1 mv to 50 mv.
